(19) United States
(12) Patent Application Publication
(10) Pub. No.: US 2024/0033011 A1
(43) Pub. Date: Feb. 1, 2024

(54) METHOD FOR DETERMINING PUNCTURE ENTRY LOCATION USING FLUOROSCOPY

(71) Applicant: Boston Scientific Medical Device Limited, Galway (IE)

(72) Inventors: Lauren Koon, Mississauga (CA); Gareth Davies, Toronto (CA)

(21) Appl. No.: 18/361,021

(22) Filed: Jul. 28, 2023

Related U.S. Application Data
(60) Provisional application No. 63/369,692, filed on Jul. 28, 2022.

(51) Int. Cl. A61B 34/20; A61B 17/00

(52) U.S. Cl. CPC A61B 34/20; A61B 17/00234; A61B 2017/00247; A61B 2034/2055

(57) ABSTRACT

A method and apparatus are disclosed for ensuring safe dilation after puncture. The method includes puncturing the target tissue via a distal tip with the puncturing device. The flexible puncturing device is advanced through the puncture into a cardiac space and visualizing the flexible puncturing device using a visualization system. The user checks the constraints imposed on the flexible puncturing device within the new cardiac space.

### CROSS-REFERENCES TO RELATED APPLICATIONS

This application claims the benefit of U.S. Provisional Application No. 63/369,692, entitled “METHOD FOR DETERMINING PUNCTURE ENTRY LOCATION USING FLUOROSCOPY,” and filed Jul. 28, 2022, which is hereby incorporated by reference in its entirety.

### TECHNICAL FIELD

The disclosure relates to a method of identifying a cardiac space using a flexible puncturing device. Specifically, the flexible puncturing device is advanced into the cardiac space and visualized using fluoroscopy prior to dilating the puncture site or space, thereby preventing the risk of inadvertent dilation.

### BACKGROUND OF THE ART

Known medical devices are configured to withdraw pressure and/or inject contrast to determine if puncture was successful prior to dilation.

### SUMMARY

Example 1 is a method of ensuring safe dilation after puncture of a tissue. The method includes puncturing the tissue with a distal tip of a flexible puncture device at a target location. The method includes advancing the flexible puncturing device through the puncture into a cardiac space defined by a heart structure. The method includes visualizing the flexible puncturing device using a visualization system, and checking for constraints imposed on the flexible puncturing device by the heart structure.

Example 2 is the method of Example 1, wherein the step of checking for constraints includes checking if the flexible puncturing device has been deflected by an atrial wall.

Example 3 is the method of Example 1, wherein the step of checking for constraints includes checking if the flexible puncturing device has been deflected by an aorta wall.

Example 4 is the method of Example 3, wherein further advancement of the flexible puncturing device causes the flexible puncturing device to advance superiorly into an aorta.

Example 5 is the method of Example 1, wherein the step of checking for constraints includes checking if the flexible puncturing device has been deflected by a wall defining a pericardial space.

Example 6 is the method of Example 5, wherein further advancement of the flexible puncturing device causes the flexible puncturing device to be advanced within the pericardial space and around a surface of a heart.

Example 7 is the method of Example 1, wherein the step of checking for constraints includes rotating the flexible puncturing device and checking the rotation on a visualization system.

Example 8 is the method of Example 1, wherein the visualization system includes fluoroscopy.

Example 9 is the method of Example 1, wherein the flexible puncture device is a wire, guidewire, or microcatheter.

Example 10 is the method of Example 1, wherein the flexible puncturing device includes an energy delivery device located at a distal tip thereof.

Example 11 is the method of Example 1, wherein the flexible puncturing device includes a sharp distal tip.

Example 12 is the method of Example 1, wherein the flexible puncturing structure includes at least one radiopaque marker.

Example 13 is the method of Example 1, further comprising advancing a dilator over the flexible puncturing device in order to dilate the puncture.

Example 14 is a method of puncturing tissue. The method includes puncturing the tissue at a target location with a distal tip of a flexible puncture device. The method includes advancing the flexible puncturing device through the puncture into a cardiac space defined by a heart structure. The method includes manipulating the flexible puncturing device in the cardiac space. The method includes visualizing the flexible puncturing device using a visualization system, and checking for constraints imposed on the flexible puncturing device by the heart structure.

Example 15 is the method of Example 14, wherein manipulating the flexible puncturing device includes rotating the flexible puncturing device, advancing the flexible puncturing device, or retracting the flexible puncturing device.

Example 16 is a method of Example 14, wherein the flexible puncturing device includes a distal curve portion.

Example 17 is a method of Example 16, wherein the distal curve portion is configured to assume a 3D structure.

Example 18 is the method of Example 16, wherein the step of checking for constraints includes checking if the flexible puncturing device has been deflected by an atrial wall, an aorta wall, or a wall defining a pericardial space.

Example 19 is the method of Example 14, wherein the flexible puncturing structure includes at least one radiopaque marker.

Example 20 is a method of dilating tissue. The method includes puncturing the tissue at a target location with a distal tip of a flexible puncture device. The method includes advancing the flexible puncturing device through the puncture into a cardiac space defined by a heart structure. The method includes manipulating the flexible puncturing device in the cardiac space. The method includes visualizing the flexible puncturing device using a visualization system. The method includes checking for constraints imposed on the flexible puncturing device by the heart structure, and advancing a dilator over the flexible puncturing device in order to dilate the puncture.

### BRIEF DESCRIPTION OF THE DRAWINGS

In order that the invention may be readily understood, embodiments of the invention are illustrated by way of examples in the accompanying drawings, in which:

FIG. 1 depicts side views of an embodiment of a tissue puncturing system.

FIG. 2 depicts a side view of an embodiment of a tissue puncturing device of the tissue puncturing system illustrated in FIG. 1.

FIG. 3 depicts a side view of an embodiment of a tissue puncturing device of the

FIG. 4 depicts a view of an embodiment of the tissue puncturing device of FIG. 1 within a left atrium of a heart.

FIG. 5 depicts a view of an embodiment of the tissue puncturing device of FIG. 1 within a left pulmonary vein of a heart.

FIG. 6 depicts a view of an embodiment of the tissue puncturing device of FIG. 1 within an aorta of a heart.

FIGS. 7 and 8 depict a view of an embodiment of the tissue puncturing device of FIG. 1 within a pericardial space of a heart.

FIG. 9 depicts a view of an embodiment of the tissue puncturing device of FIG. 1 within a right atrium of a heart.

FIGS. 10A-10C depict an embodiment of a tissue puncturing device with a 3D distal structure (FIG. 10A), a front view of the tissue puncturing device within a left atrium (FIG. 10B), and a side view of the tissue puncturing device within a left atrium (FIG. 10C).

FIG. 11 depicts the embodiment of the tissue puncturing device of FIG. 10A within an aorta of a heart.

FIGS. 12A-12C depict an embodiment of the tissue puncturing device within a left atrium (FIG. 12A), an aorta (FIG. 12B), and a pericardial space within a heart (FIG. 12C).

FIGS. 13A-13E depict an embodiment of the tissue puncturing device of FIG. 1 being rotated within a free chamber from 0° to 180°.

FIGS. 14A-14E depict an embodiment of the tissue puncturing device of FIG. 1 being rotated within a constrained chamber from 0° to 180°.

### DETAILED DESCRIPTION

Many diseases and disorders, such as, for example, heart failure, atrial fibrillation, mitral valve disease, and others, specifically impact or are addressable in the left side of the heart. Accordingly, many interventional percutaneous cardiac procedures require access from one internal space to another through tissues within the human body. For example, cardiac procedures require creating punctures to access different chambers or spaces within a patient's heart. In a specific example, a transseptal puncture procedure involves creating a puncture between the right atrium and the left atrium of a patient to deliver end therapies, such as left atrial appendage occlusion procedures, mitral valve repair and replacement procedures, atrial shunt procedures, and many more. In additional to therapeutic interventional procedures, indications for access to the left side of the heart also include diagnostic procedures, including, for example, hemodynamic measurements (e.g., left atrial pressure, trans-mitral pressure gradient, etc.).

In further examples, a puncture site may be created to provide access to ventricular chambers, the pericardial space, and other heart structures. In these procedures, the puncture site may undergo dilation to enlarge the diameter (for example, using a dilator to enlarge the puncture site during a transseptal procedure). However, complications arise when a puncture site is formed and subsequently dilated in an unintended location or site, for example a hemopericardium (which occurs when blood accumulates in the pericardial sac resulting in an increase in pressure within the sac). These complications often lead to serious surgeries, injuries, and may even result in death. The inadvertent puncture of a site/location may pose relatively mild complications compared to the subsequent dilation of the inadvertent puncture location. If the puncture site is in an incorrect location, the puncture formed within the tissue is relatively small. However, when the puncture is further dilated to a larger diameter, more serious complications arise.

For a needle-based solution or puncture, physicians rely on pressure readings and/or contrast injections. Pressure readings are used to identify the location of the distal tip of the needle. An external pressure transducer is connected to the lumen of the needle via a Luer at the proximal end/handle. The pressure reading collected by the external pressure transducer varies depending on the location of the distal tip of the needle. Different chambers/locations within the heart have different characteristic waveforms. However, pressure readings with fluid medical transducers are unreliable and prone to error with movement. An alternative method is the injection of contrast which is visible under fluoroscopy. Contrast can be used to identify heart structures during a procedure (for example, the tenting of the fossa ovalis during a transseptal procedure) and to identify the accessed space once the puncture has been completed. However, in some cases, the use of contrast may be harmful to patients.

In addition, the use of pressure readings and contrast injections are not feasible for puncture devices which do not have a lumen, for example some wire-based solutions. Using a wire-based solution provides advantages over a traditional needle-based solution as the puncturing device also provides the advantages of anchoring and acting as a guiderail for the ancillary devices, thereby removing the need for a separate guidewire and reducing the number of steps for completing procedures.

As such, there exists a need for identifying an accessed cardiac space after puncturing with a wire-based solution which solves the problems associated with the needle-based solutions. The ability to identify the accessed cardiac space would allow physicians to identify any potential risks associated with the subsequent dilation of the puncture site.

In one broad aspect, embodiments of the present disclosure provides a method of ensuring safe dilation of a site after puncture. The method includes the steps of puncturing a target tissue via a distal tip of a flexible puncturing device; advancing the flexible puncturing device through the puncture site into a desired cardiac space; visualizing the flexible puncturing device using a visualization system; and, checking for constraints imposed on the flexible puncturing device within the cardiac space. The constraints may include deflection of the puncture device by an atrial wall, an aorta wall, or while advancing the puncture device from superior or inferior route, or while advancing into a pericardial space, and/or around a surface of a heart.

Another feature, separate or in combination with the previous steps, is a step of rotating the flexible puncturing device and checking the rotation on a visualization system.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. It is to be understood that when referring to various directions, such as “left” and “right” that the terminology is with reference to anatomical left and anatomical right.

The method of the present disclosure utilizes a flexible puncturing device. The present method may be applicable to wires and/or microcatheters which are configured to puncture a tissue within the body. Furthermore, even though the application described herein references cardiac procedures, it is to be understood that this method could be applied to any medical procedure where a physician is creating a puncture from one location to another where fluoroscopy is used.

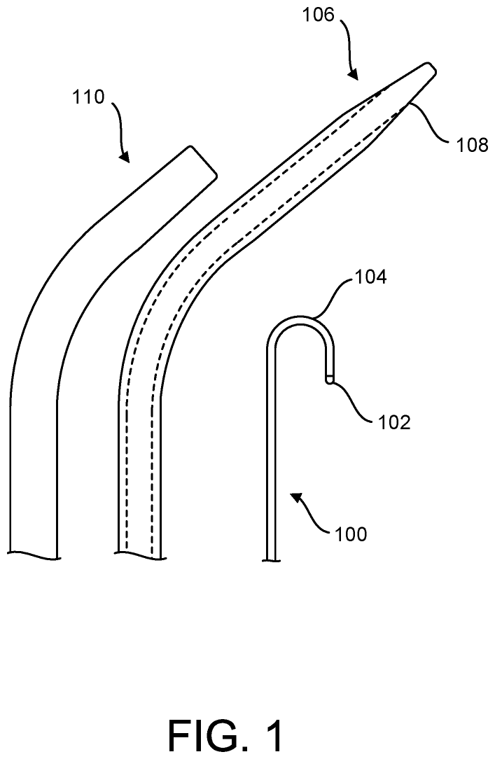

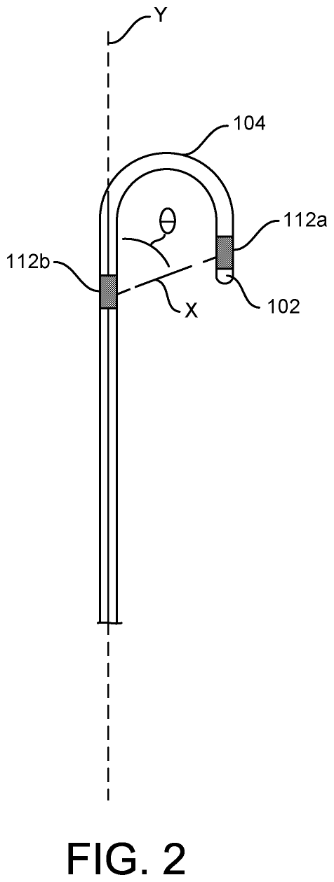

With reference to FIG. 1, a system configured to puncture tissue is shown. In some embodiments, the system may include a flexible puncturing device 100 having a low-profile (e.g., small diameter, small cross-sectional area, etc.). A low-profile device should form a small puncture which would not be large enough to result in hemodynamic consequences. In other words, the low-profile of the device would create a puncture with a small cross-section resulting in minimal fluid flow. Additionally, having a low-profile would allow the device 100 to be withdrawn/retracted without dilation if an incorrect puncture site has been made. Further, the profile of the puncturing device 100 should enable ancillary devices to be tracked overtop. Some examples of the flexible puncturing device may include a wire, guidewire, or a microcatheter, configured to puncture a tissue. In one embodiment, the flexible puncturing device 100 is capable of delivering energy to a target tissue, forming a puncture. In this embodiment, the flexible puncturing device 100 comprises an energy delivery device (e.g., an electrode) at the distal tip 102, operable to deliver energy from a generator (e.g., a radiofrequency generator). The distal tip 102 may be atraumatic, for example a dome, hemisphere, cylinder, etc., reducing pressure exerted on the target tissue and preventing inadvertent puncture or damage/trauma to surrounding tissue structures. The flexible puncture device 100 may further include a distal curve portion 104, such as a J-shape, pigtail, spiral, or a 3D configuration, to provide anchoring and an atraumatic surface (i.e., an atraumatic leading edge) when contacting surrounding tissue. The flexible puncturing device 100 is further configured to be visualized on a visualization system, such as fluoroscopy. The entire flexible puncturing device 100 may be radiopaque and visualized under fluoroscopy. For example, the flexible puncture device 100 may be composed of stainless steel or nitinol. In some embodiments, the flexible puncture device 100 may include additional features to enhance visualization. For example, the flexible puncture device 100 may include a radiopaque coil positioned along, and extending the length of, the distal curve 104, and/or a radiopaque marker with the two ends (and far enough to visually identify as two differentiable points on the wire, and that the vector formed are not coaxial to the proximal rotation portion) positioned along the device 100 in order to visually identify as two points, and for identifying whether or not the device 100 is rotating. Alternatively, the flexible puncture device 100 may include a radiopaque coil positioned along, and extending the length of, the distal curve 104, and/or at least one radiopaque markers 112a, 112b positioned along the distal portion (and/or distal tip 102) of the device 100. In this alternative, the flexible puncturing device 100 may have two radiopaque markers 112a, 112b positioned such that a vector X may be created between the two markers, the vector X created may be positioned such that they are not coaxial (not in line) with the rotating axis Y of the device 100, see FIG. 2. The flexible puncturing device 100 may include any number of features enabling visualization under fluoroscopy.

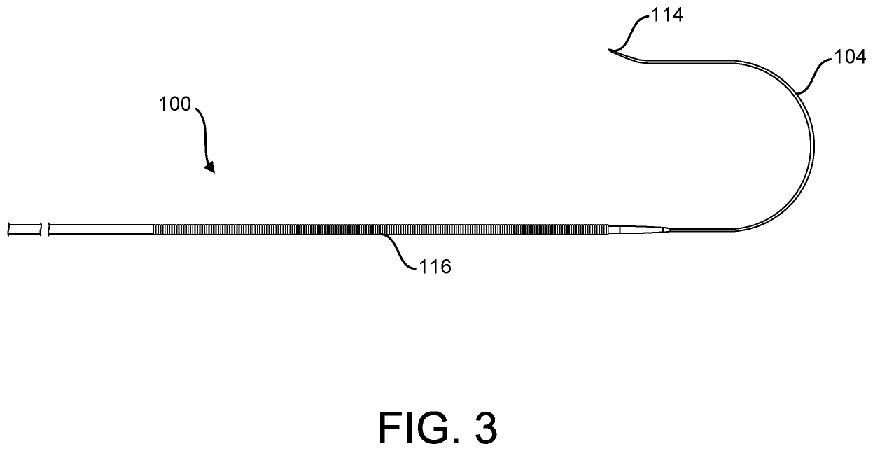

In an alternative embodiment, the flexible puncturing device 100 described above may create a puncture using a sharp tip (e.g., a mechanical puncture). An example of this embodiment is depicted in FIG. 3. In this embodiment, the distal tip 102 may include a sharp tip 114 for delivering mechanical energy to the target tissue. In this embodiment, the puncturing device 100 may be configured such that it is selectively sharp. In other words, the puncturing device 100 when constrained within the introducer assembly (e.g., dilator 106 and/or sheath 110) is configured to puncture tissue. In one embodiment, the flexible puncturing device 100 alone may have insufficient stiffness to puncture tissue. Thus, to perform a puncture, the flexible puncture device 100 may be constrained by the introducer assembly, which provides adequate stiffness, to puncture the tissue. Upon advancement of the puncturing device 100 out of the introducer assembly, the puncturing device 100 would begin to prolapse due to the insufficient stiffness, therefore becoming atraumatic. Due to this configuration, the sharp tip 114 would only puncture a small thickness of adjacent tissue (that is, tissue adjacent the introducer assembly) before assuming an atraumatic configuration, thereby preventing unintended puncture of anatomical structures upon advancement into the second anatomical location. Alternatively, or in combination with the previous embodiment, the distal curve portion 104 of the puncturing device 100 may be configured to protect the sharp distal tip 102 from contacting tissue (e.g., a pigtail shape, 3D spiral, J-shaped, etc.) such that inadvertent damage to the surrounding tissue is mitigated. An additional benefit of having a selectively sharp puncture device 100 is that the device is sharp when constrained within the introducer assembly, thus, when the tip 114 is protruding at a maximum puncturing distance from the tip of the introducer assembly. In other words, the sharp tip 114 would assume an atraumatic configuration when advanced a large distance (beyond puncturing distance) from the distal tip of the introducer assembly. The flexible puncturing device 100 may include radiopaque markers (as previously described) and/or a radiopaque coil 116.

In a further alternative embodiment, a non-puncturing device may be utilized. In this embodiment, the system would further include a puncturing needle with a lumen, configured to receive a flexible device (e.g., guidewire) with the radiopaque characteristics described above.

The system may further include an introducer assembly. In some embodiments the introducer assembly includes a dilator 106 configured to dilate the puncture formed by the flexible puncturing device 100. The dilator 106 includes a lumen, extending the length of the dilator 106 and configured to receive the flexible puncture device 100. The distal end of the dilator 106 includes a tapered portion 108 which, upon being advanced overtop (envelops) the flexible puncture device 100, enlarges the puncture hole formed in the target tissue. The dilator 106 may be required to provide the system with sufficient stiffness and support throughout the procedure. In some embodiments, the dilator 106 may include a means for providing adequate support, stiffness, pushability, and/or torquability, to the flexible puncturing device 100. In some examples, the dilator 106 may include a reinforcing member, such as a hypotube, embedded within the dilator 106 itself. Alternatively, a separate device (usable independently from the dilator 106), such as a stylet, may be inserted into the dilator 106 lumen to provide sufficient properties (e.g., stiffness, force transmission, torquability, and/or shapeability, etc.) to the system.

With continued reference to FIG. 1, the introducer assembly may further include a sheath 110 configured to receive the dilator 106 (which receives the flexible puncturing device 100). In some embodiments, the sheath 110 may be a fixed curve sheath. In an alternative embodiment, the sheath 110 may be a steerable sheath configured with an actuator to enable steering of the system.

The method of the present invention provides a means of identifying a newly accessed space after puncturing using a wire-based solution and, therefore, identifying the risk of dilating the puncture site. The method takes advantage of characteristics of the flexible puncture device 100 (e.g., trackability into heart structures, stiffness that allows for deformation against tissues, curve retention properties, torque transfer properties, surface properties that allow for movement against tissue, etc.) and visibility under fluoroscopy. Broadly, the method involves advancing the flexible puncturing device 100 into the newly accessed space and visualizing the constraints imposed on the puncturing device 100 by the surrounding tissue structures prior to advancing the dilator 106 over of the flexible puncturing device 100.

In one embodiment, the steps of the method may include the following steps: advancing an introducer assembly, such as a dilator 106 and/or sheath 110, with a flexible puncturing device 100 to a first location; positioning the distal tip of the introducer assembly and flexible puncturing device 100 against the target tissue; puncturing the target tissue with the puncturing device distal tip 102; advancing the puncturing device 100 through the puncture and into a second location; manipulating the puncture device 100 (e.g., rotating, advancing, retracting, etc.); and visualizing (inspecting) how the flexible puncturing device 100 reacts (i.e., the constraints imposed by anatomical structures of the second location) within the second location; if the flexible puncturing device 100 is in the incorrect location the physician can retract the device from the location and re-perform the puncture; if the flexible puncture device 100 is in the correct second location, the physician may advance the dilator 106 overtop of the puncturing device 100, thereby safely dilating puncture. The sheath 110, if being used, may be advanced over the dilator 106 to provide access for ancillary or end therapy devices.

In an alternative method, a non-puncturing device, such as a guidewire, may be used. In this embodiment, the method may include the steps of: puncturing the target tissue with a puncturing tip of a needle; advancing the guidewire through the puncture and into the second location; and, manipulating the guidewire (e.g., rotating, advancing, etc.) and visualizing how the guidewire reacts (i.e., the constraints imposed by anatomical structures of the second location) within the second location.

The following provides examples of cardiac access procedure, but it should be appreciated that similar techniques may be used for any procedures which involve creating and dilating a puncture site from a first location to a second location. Some examples of alternative procedures may include crossing the coronary sinus wall into the left atrium or left ventricle, crossing from one ventricle to the other (e.g., right ventricle to left ventricle), or other interventional procedures outside of cardiology such as moving from a tissue wall into a vessel, crossing occlusions, etc.

As enumerated above, the heart structures of interest during a transseptal puncture procedure includes: the right atrium, left atrium, aorta, and pericardial sac. During a transseptal puncture, the right atrium is the first location and is an open chamber (free space). The left atrium is the preferred second location during a transseptal puncture procedure and is an open chamber (free space). The aorta is a major vessel positioned anterior to the fossa ovalis (on the atrial septum of a heart) and travels inferiorly (towards the aortic valve) and superiorly (towards the aortic arch). The vessel is considered a closed space as there is little room for lateral movement. The pericardial space is a small space (<2 mm) between the epicardium and the pericardial sac, with very little lateral space movement, and follows the surface of the heart.

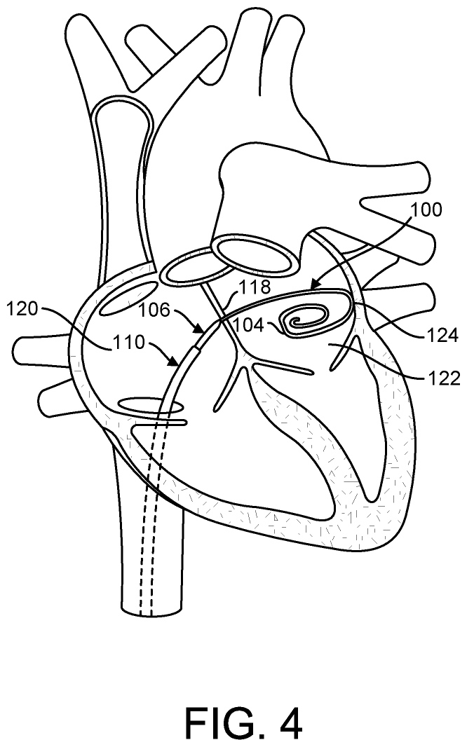

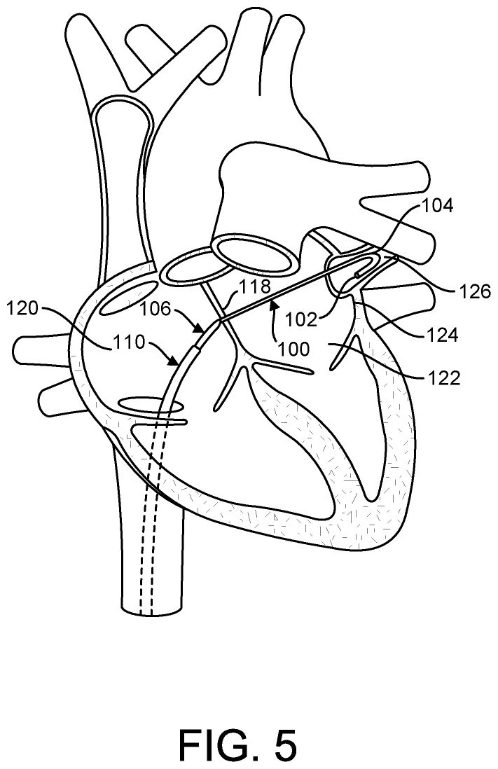

During a trans septal procedure, it is desirable to create a puncture in the atrial septum 118 (FIG. 4), specifically the fossa ovalis, thereby gaining access to the left atrium 122 from the right atrium 120. To confirm left atrium 122 access after puncture, the physician may manipulate the flexible puncturing device 100 by advancing the flexible puncture device 100 through the puncture site and visualize the movement and/or constraints imposed on the flexible puncturing device 100. For example, if the flexible puncturing device 100 has successfully punctured the fossa ovalis, it would travel across the fossa ovalis into the left atrium 122. Further advancement of the flexible puncturing device 100 would result in the puncturing device 100 contacting the lateral left atrial wall 124, thereby causing the puncturing device 100 to prolapse or deflect (and, in some instances, assume the unconstrained curved distal portion 104 configuration), as seen in FIG. 4. Alternatively, the flexible puncturing device 100 may enter one of the left pulmonary veins 126. In this instance, the flexible puncturing device 100 would move into the left pulmonary vein 126, as seen in FIG. 5. When a physician observes these conditions on the flexible puncturing wire 100 under fluoroscopy, the physician can be confident that the puncture was successful and advance the dilator 106 and/or sheath 110 through the puncture.

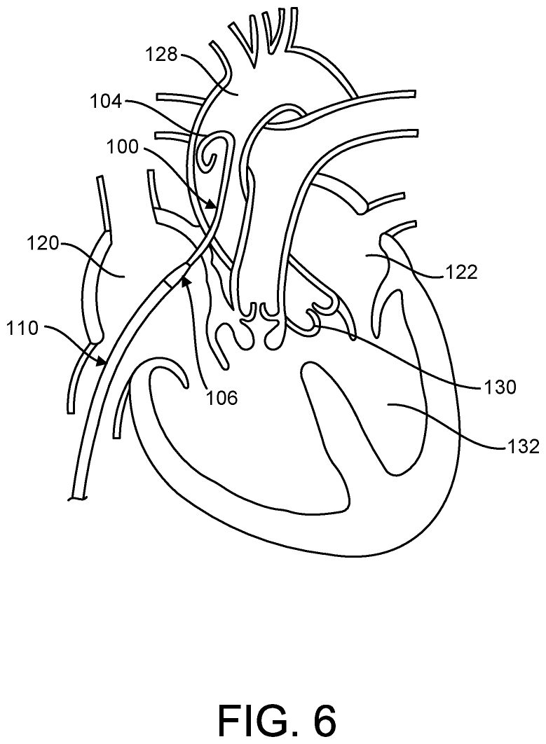

In the event where the puncturing device 100 has entered the aorta 128, near the root, the physician may advance the flexible puncturing device 100 and would observe that the puncturing device 100 advances superiorly towards the aortic arch and continues to follow that character profile, as depicted in FIG. 6, or in some instances it may enter the head/neck arteries (e.g., the brachiocephalic, common carotid, or subclavian arteries). Alternatively, the flexible puncturing device 100 may puncture into the aorta and advance inferiorly towards the aortic valve 130 or pass through the left ventricle 132. If the physician observes these constraints imposed on the flexible puncturing device 100, the physician can determine that the puncture is unsuccessful. The physician may then retract the puncturing device 100, reposition, and perform the puncture again.

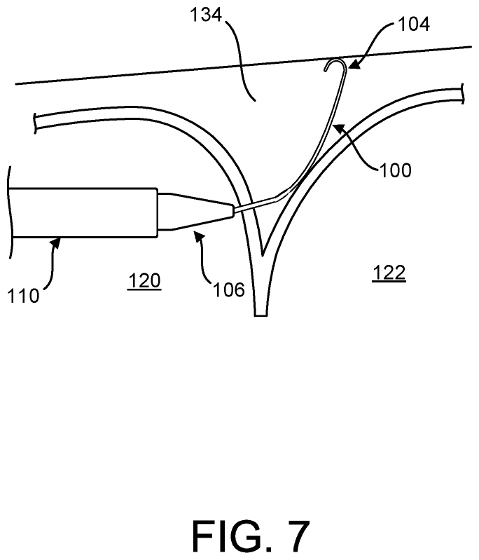

In another situation, the puncturing device 100 may perforate into the pericardial space 134, for example FIG. 7. In this situation, advancement of the flexible puncturing device 100 would result in the device 100 wrapping around the cardiac profile as it tracks within the pericardial space 134, as depicted in FIG. 8. This movement is an indicator of epicardial access and, as such, the physician can retract the puncturing device 100 back into the right atrium 120, reposition, and perform the puncture again.

In some instances, the physician may incorrectly puncture through, beyond the pericardium and into the thoracic cavity. In this situation, advancement of the flexible puncturing device 100 would depend on where the puncture occurred. For example, advancement may result in the flexible puncturing device 100 being advanced between the lung and parietal pleura or along the diaphragm on the inferior edge. In these examples, there would be no sizeable fluid filled chambers and the physician would know something is a miss by the tactile feel of the advancement, non-linear advancement, or deformation of the wire. Alternatively, in some cases, under fluoroscopy the flexible puncturing device 100 may appear similar to the pericardial space profile. These features would indicate to the physician that the thoracic cavity has been punctured, the physician can retract the puncturing device 100 back into the right atrium 120, reposition, and perform the puncture again.

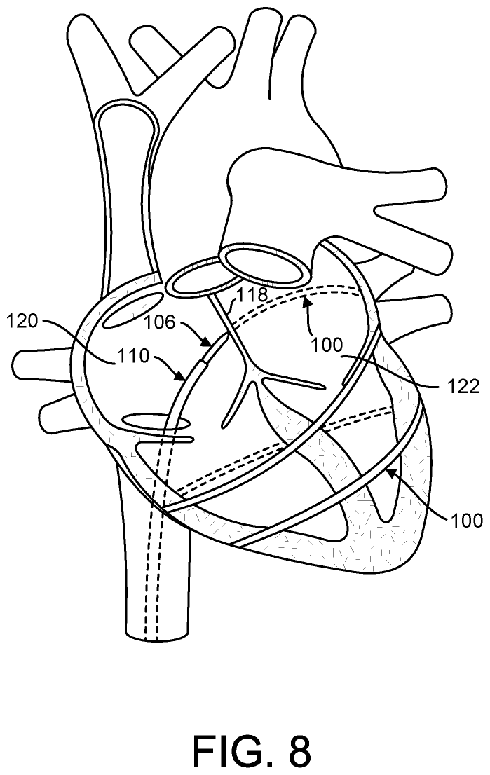

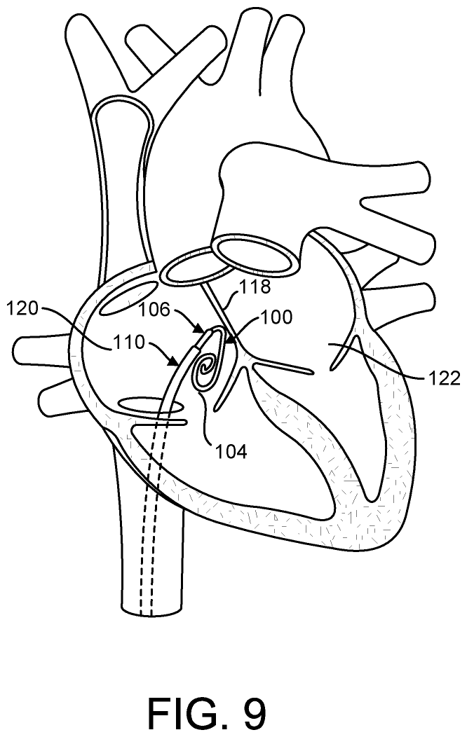

A physician may also determine that if the puncture was unsuccessful in crossing the atrial septum 118, thus having the puncturing device 100 remaining in the right atrium 120. In this situation, advancement of the flexible puncturing device 100 out of the introducer assembly would result in the puncturing device 100 deflecting off the (unpunctured) atrial septum 118 and assume the unconstrained configuration within the right atrium 120 (see FIG. 9). If the physician observes these constraints imposed on the flexible puncturing device 100, the physician can determine that the puncture is unsuccessful. The physician may then retract the puncturing device 100, reposition, and perform the puncture again.

In addition to, or alternative to, manipulating the puncturing device 100 through advancement, the physician may determine the chamber volume of the second location by observing the constraints imposed on the distal curve portion 104 within the space. In other words, the physician can observe (if any) shape deformation of the configuration of the distal curve portion 104 to determine volume characteristics. In an open chamber, the distal curve portion 104 would assume the expected (natural), unconstrained (normal), configuration. In contrast, in an enclosed chamber the distal curve portion 104 would be deformed by structures and may present an unexpected shape (e.g., if in a smaller chamber, the distal curve portion 104 may be wedged between structures, displaying a shape that would look flattened). In some embodiments, the distal curve portion 104 may assume a 3D structure. In alternative embodiments, the distal curve portion 104 may assume a 2D structure which, when visualized in an enclosed chamber, the distal curve portion 104 may appear shortened. In some embodiments, it may be desirable to view the configuration under more than one fluoroscopic view or angle for confirmation.

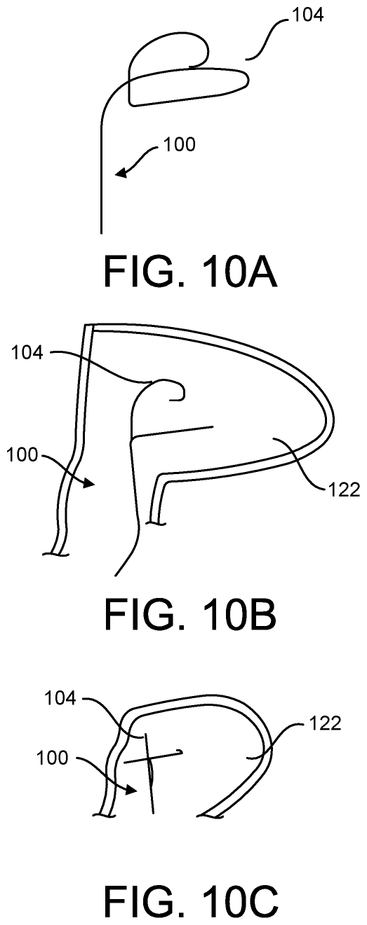

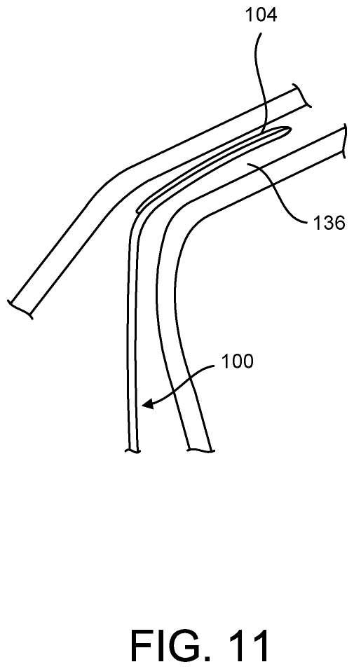

For example, if the distal curve portion 104 of the flexible puncturing device 100 comprises a characteristic 3D curve, upon being advanced into the left atrium 122 (open chamber), the distal curve portion 104 would assume the unconstrained 3D shape (FIG. 10A-10C). When a physician observes these conditions on the flexible puncturing wire 100, the physician can be confident that the puncture was successful and advance the dilator 106 and/or sheath 110 through the puncture. If the flexible puncturing device 100 has entered the aorta (enclosed chamber), the distal curve portion 104 would look flattened as it is wedged within the vessel 136 (FIG. 11). Alternatively, if the physician has entered the thoracic cavity (e.g., between the lung and parietal pleura or along the diaphragm on the inferior edge), the distal curve portion 104 would look flattened. If the physician observes these constraints imposed on the flexible puncturing device 100, the physician can determine that the puncture is unsuccessful. The physician may then retract the puncturing device 100, reposition, and perform the puncture again.

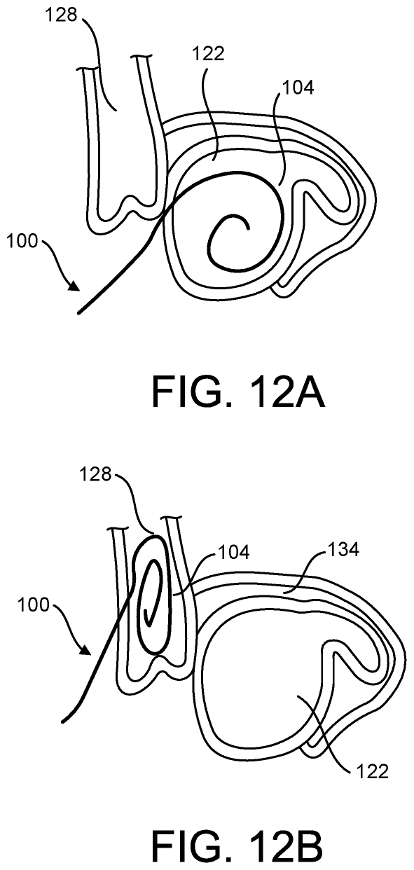

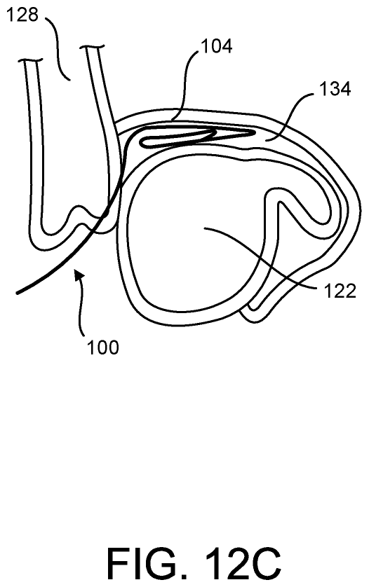

In a further alternative embodiment, the distal curve portion 104 of the flexible puncturing device 100 may be comprised of a shape memory material (e.g., nitinol). The shape memory material may be limited to the distal curve portion 104 or may comprise the entire length of the flexible puncture device 100. In this embodiment, the distal curve portion 104 may be formed such that it is similar in size and/or shape as the target/desired location (e.g., the left atrium in a transseptal puncture). Alternatively, the distal curve portion 104 may be formed to fit/fill the target space when unconstrained. For theses embodiments, the physician would be able to determine, when viewed under any suitable imaging, if the device 100 has undergone deformation or not which will inform the physician if device has entered the target location successfully, as seen in FIG. 12A. If there is deformation, the device 100 is in a non-target location, as seen in FIG. 12B-12C. If there is no deformation, the device 100 has entered the target location.

In addition to, or alternative to, manipulating in the flexible puncturing device 100 as previously described, a physician may advance the flexible puncturing device 100 into the second location and rotate the device 100 within the space. The flexible puncturing device 100 may comprise a single or multiplanar distal curve 104 which is visible under fluoroscopy. If the device 100 is constrained by tissue, the device 100 may not rotate or may get caught during the rotation (this may be visualized as the device whips or snags). If the device is unconstrained by the tissue, the device 100 would rotate freely.

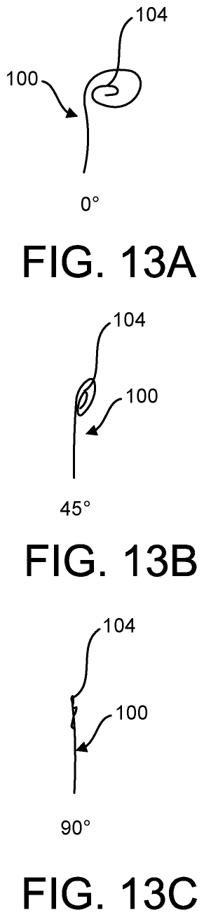

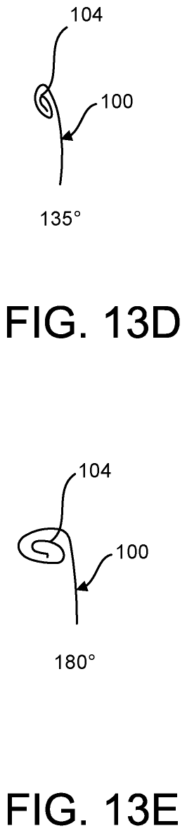

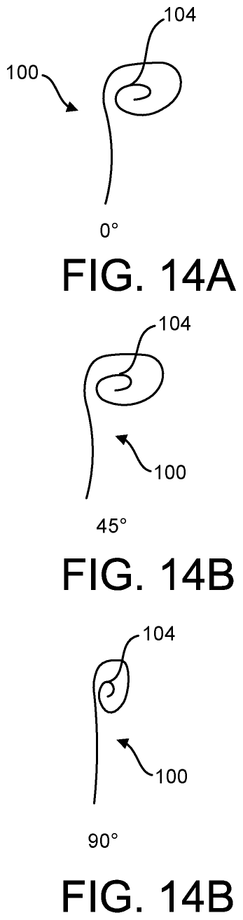

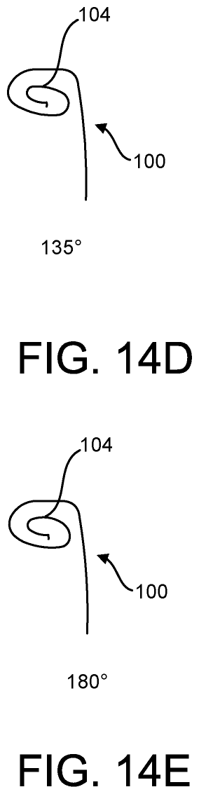

For example, if the physician is trying to determine if the device 100 has punctured successfully into the left atrium 122, the physician may advance the flexible puncturing device 100 through the puncture site and rotate the device 100. If the device 100 rotates freely (see FIG. 13A-13E, which shows rotation from 0° to 180°), this would be an indicator to the physician that the device has entered the left atrium 122 and the physician can be confident that the puncture was successful and advance the dilator 106 and/or sheath 110 through the puncture. If the device 100 gets snagged or whips around, as seen in FIG. 14A-14E (which shows rotation from 0° to 180°), this may be an indicator that the device 100 has punctured into a vessel, thoracic cavity, or a non-target location and the puncture is unsuccessful. The physician may then retract the puncturing device 100, reposition, and perform the puncture again.

In some embodiments, the rotation of the flexible puncturing device 100 may be viewed on a plurality of static images, for example through “cine” fluoroscopy. In cine fluoroscopy, the capture can be simultaneous with the torquing movement. Alternatively, an image may be captured after each incremental rotation of the device 100.

In one embodiment, a fixed curve shape of a puncturing device 100 may be used. In this embodiment, the flexible puncturing device 100 may be configured to retain the distal curve 104 shape. Additionally, there should be sufficient torque-ability and/or compatibility with a torque device. The flexible puncturing device 100 may include a radiopaque feature along the distal portion 104. This may be limited to just the distal portion 104 or extend along the entire device 100. In some embodiments, the radiopaque feature may be a coil and/or via radiopaque markers. As an example, a device 100 may comprise at least two radiopaque markers positioned along the device 100 such that a vector may be created between the two markers which is offset (e.g., not coaxial) with the rotating axis.

The embodiment(s) of the invention described above is(are) intended to be exemplary only. The scope of the disclosure is therefore intended to be limited solely by the scope of the appended claims.

It is appreciated that certain features of the disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims.

We claim:

1. A method of ensuring safe dilation after puncture of a tissue, the method comprising the steps of:
puncturing the tissue with a distal tip of a flexible puncture device at a target location;
advancing the flexible puncturing device through the puncture into a cardiac space defined by a heart structure;
visualizing the flexible puncturing device using a visualization system; and,
checking for constraints imposed on the flexible puncturing device by the heart structure.

2. The method of claim 1, wherein the step of checking for constraints includes checking if the flexible puncturing device has been deflected by an atrial wall.

3. The method of claim 1, wherein the step of checking for constraints include checking if the flexible puncturing device has been deflected by an aorta wall.

4. The method of claim 3, wherein further advancement of the flexible puncturing device causes the flexible puncturing device to advance superiorly into an aorta.

5. The method of claim 1, wherein the step of checking for constraints includes checking if the flexible puncturing device has been deflected by a wall defining a pericardial space.

6. The method of claim 5, wherein further advancement of the flexible puncturing device causes the flexible puncturing device to be advanced within the pericardial space and around a surface of a heart.

7. The method of claim 1, wherein the step of checking for constraints includes rotating the flexible puncturing device and checking the rotation on a visualization system.

8. The method of claim 1, wherein the visualization system includes fluoroscopy.

9. The method of claim 1, wherein the flexible puncture device is a wire, guidewire, or microcatheter.

10. The method of claim 1, wherein the flexible puncturing device includes an energy delivery device located at a distal tip thereof.

11. The method of claim 1, wherein the flexible puncturing device includes a sharp distal tip.

12. The method of claim 1, wherein the flexible puncturing structure includes at least one radiopaque marker.

13. The method of claim 1, further comprising advancing a dilator over the flexible puncturing device in order to dilate the puncture.

14. A method of puncturing tissue, the method comprising the steps of:
puncturing the tissue at a target location with a distal tip of a flexible puncture device;
advancing the flexible puncturing device through the puncture into a cardiac space defined by a heart structure;
manipulating the flexible puncturing device in the cardiac space;
visualizing the flexible puncturing device using a visualization system; and,
checking for constraints imposed on the flexible puncturing device by the heart structure.

15. A method of claim 14, wherein manipulating the flexible puncturing device includes rotating the flexible puncturing device, advancing the flexible puncturing device, or retracting the flexible puncturing device.

16. A method of claim 14, wherein the flexible puncturing device includes a distal curve portion.

17. A method of claim 16, wherein the distal curve portion is configured to assume a 3D structure.

18. The method of claim 16, wherein the step of checking for constraints includes checking if the flexible puncturing device has been deflected by an atrial wall, an aorta wall, or a wall defining a pericardial space.

19. The method of claim 14, wherein the flexible puncturing structure includes at least one radiopaque marker.

20. A method of dilating tissue, the method comprising the steps of:
puncturing the tissue at a target location with a distal tip of a flexible puncture device;
advancing the flexible puncturing device through the puncture into a cardiac space defined by a heart structure;
manipulating the flexible puncturing device in the cardiac space;
visualizing the flexible puncturing device using a visualization system;
checking for constraints imposed on the flexible puncturing device by the heart structure; and
advancing a dilator over the flexible puncturing device in order to dilate the puncture.
